(19) United States
(12) Patent Application Publication
(10) Pub. No.: US 2023/0086469 A1
(43) Pub. Date: Mar. 23, 2023

(54) AUTOANTIGENIC PEPTIDES (CALVICIFIV), PRESENTED BY TOLEROGENIC DENTRITIC CELLS, USEFUL FOR THE PERSONALIZED TREATMENT OF RHEUMATOID ARTHRITIS

(71) Applicant: UNIVERSIDAD DE CHILE, Santiago (CL)

(72) Inventors: Juan Carlos Aguillón Gutiérrez, Santiago (CL); Diego Francisco Catalán Martina, Santiago (CL); Lilian Andrea Soto Saez, Santiago (CL); Jaxaira Amilene Maggi Orellana, Santiago (CL); Octavio Alexis Aravena Madariaga, Santiago (CL); Katina Schinnerling, Santiago (CL)

(21) Appl. No.: 17/638,770

(22) PCT Filed: Aug. 30, 2019
(86) PCT No.: PCT/IB2019/057357

§ 371 Date: Feb. 25, 2022

(51) Int. Cl. A61K 39/00; A61K 39/39; A61P 37/06; A61K 31/519; A61K 31/52; A61K 31/4706; A61K 38/13; A61K 31/65; A61K 31/42; A61K 31/5383; A61K 31/655; A61K 33/242; A61K 39/395

(52) U.S. Cl. CPC A61K 39/0008; A61K 39/39; A61P 37/06; A61K 31/519; A61K 31/52; A61K 31/4706; A61K 38/13; A61K 31/65; A61K 31/42; A61K 31/5383; A61K 31/655; A61K 33/242; A61K 39/3955; A61K 2039/5154; A61K 2039/55572

(57) ABSTRACT

The present invention provides an immunomodulatory composition useful for treating or preventing joint damage comprising at least a set of peptides possessing an amino acid sequence having at least 80%, 85% and 90% sequence identity with the peptides corresponding to SEQ ID NO:1, SEQ ID NO:2, SEQ ID NO:3 and SEQ ID NO:4 and a method for the treatment or prevention of joint damage comprising the steps of a) extract monocytes from a patient with a rheumatological disease; b) culture the monocytes extracted in the previous step in AIM-V medium with GM-CSF and IL-4; c) wash the monocytes and add dexamethasone; d) load the tDCs with the immunomodulatory composition comprising autoantigenic peptides; e) add MPLA; and f) incorporate the tDCs loaded with autoantigenic peptides into the patient. The present invention includes methods for the treatment or prevention of rheumatological disease comprising a wide range of tDCs performed by different protocols.

### FIELD OF THE INVENTION

The present invention relates generally to immunomodulatory agents useful for the treatment or prevention of rheumatological diseases and joint damage. More particularly, the present invention relates to an immunomodulatory composition comprising a set of appropriate autoantigenic peptides derived from synovial proteins, and a method for the treatment or prevention of joint damage and rheumatological diseases that consists to an antigen-specific therapy in a subject in need thereof, comprising tolerogenic dendritic cells (tDCs) pulsed with the appropriate autoantigenic peptides derived from synovial proteins to restore self-tolerance in patients with Rheumatoid Arthritis (RA), Juvenile Idiopathic Arthritis (JIA), Juvenile Rheumatoid Arthritis (JRA) and other rheumatological related diseases.

### BACKGROUND OF THE INVENTION

Rheumatoid arthritis (RA) is an incurable, disabling autoimmune disease that decreases life quality and expectancy of patients who suffer from it, there being at least 73 million people worldwide. RA is the most common form of arthritis, affecting between 0.1% and 2% of the world population. It is a disease that produces synovial hyperplasia and synovitis and, in most patients, progresses to the destruction of cartilage and joint bone, considering it as one of the main causes of disability. Relevant for the development of better therapies is the fact that most of the joint and bone destruction occurs during the first years of evolution of the disease, having been shown that more than half of the patients have bone erosions at 2 years of diagnosis, and up to 93% of patients with less than 2 years of diagnosis can have radiographic alterations, leaving 50% of these, disabled at 10 years. The current treatment of RA includes nonsteroidal anti-inflammatory drugs (NSAIDs), glucocorticoids, disease-modifying antirheumatic drugs (DMARDs) and biological agents. Although NSAIDs are only a symptomatic alternative, glucocorticoids and DMARDs are capable of interfering with the progression of the disease but producing adverse effects when administered for long periods. Within conventional DMARDs, methotrexate is the most commonly used drug, however, it has limited efficacy, with an average of 55% for the ACR20 improvement response established by the American College of Rheumatology, and also generates toxicity problems, highlighting that 42% of treated patients must abandon the therapy due to the presence of important adverse effects, adding to this, a limited rate of adherence. To overcome these important difficulties, in the last two decades biological agents have been introduced with the aim of inhibit specific pathways or targets involved in RA. However, a significant percentage of patients remain refractory to these therapies, having been reported that 20 to 40% of them do not respond to treatment. Also, even when biological therapies have a lower toxicity profile than conventional DMARDs, they are not exempt from causing severe complications such as reactivation of latent infections, possible development of autoimmunity and triggering of neoplasms. However, although these therapies can achieve remission in the majority of cases, this is generally temporary, with patients confined to receiving drugs for life, with the progressive loss of therapeutic efficacy, investing a large sum of money. In the last two decades biological agents have been introduced with the idea of inhibiting specific pathways or targets involved in RA, being approved for treatment antibodies and recombinant soluble receptors blocking cytokines, chimeric molecules that interfere with the activation of T-cells, and B-cell depleting antibodies. However, a significant percentage of patients remain refractory to these therapies, having been reported that 20 to 40% of patients with RA do not respond to treatment with tumor necrosis factor (TNF) antagonist antibodies must change to blocking antibodies of other cytokines such as interleukin (IL)-6 or B lymphocyte depletory. Also, even when biological therapies have a lower toxicity profile than cDMARDs, they are not exempt from causing severe complications such as reactivation of latent infections, possible development of autoimmunity and the triggering of neoplasms. It has been shown that in RA, DCs, together with T cells, macrophages, neutrophils and B cells, are part of the massive leukocyte infiltrate to the main target tissue, synovial tissue. It has also been shown that DCs, in their activated or mature immunogenic state (mDCs), can initiate and amplify the magnitude and intensity of a normal or pathological immune response, as in RA, while in their tolerogenic state (tDCs), can induce peripheral tolerance, silencing a normal immune response to the elimination of the aggressor, or quenching an exacerbated pathological response as in autoimmunity.

Dendritic cells (DCs) play a fundamental role in the regulation of autoimmunity and tolerance induction, so they have become a promising tool for the immunotherapy of autoimmune diseases such as RA. Several studies of autoimmunity in animal models suggest that tolerogenic dendritic cells (tDCs) are able to suppress inflammation and induce tolerance. The use of tDCs as therapy is an emerging research area that began after a visionary study, in which autologous tDCs pulsed with an influenza antigen were injected into healthy individuals, observing a tolerizing effect. Ten years after this study, the first clinical trial was published applying tDCs in patients with type 1 diabetes, demonstrating that its administration was not only safe and well tolerated, but also increased the frequency of a potentially beneficial B cell population. Thus, the US patent US 20070172453 discloses compositions and methods for the treatment and prevention of type 1 diabetes consisting of a composition for reducing an immune response against islet beta cells in a subject comprising an IGF-2 peptide. Similarly, WO 2013/138871 relates to the use of an aggrecan polypeptide including citrullinated forms thereof to treat or prevent joint damage in subjects with early RA or incipient RA; however, this patent focuses on the generation of tolerance to a single protein related to the disease, which decreases the effectiveness of the treatment and immuno-modulation, which restricts the coverage of the therapy to a certain type of patients. Something important to consider in the application of tDCs in complex autoimmune diseases such as RA, is that one of the main obstacles is the correct choice of autoantigenic immunodominant peptides to load the tDCs in order to establish an autoantigen-specific tolerance. These autoantigenic peptides must be diverse and allow to cover a greater range of sensitive population. Thus, there is an urgent need for therapies capable of restoring tolerance to self-antigens and achieving permanent remission of the disease, including immunodominant antigenic determinants suitable for the proper development of therapy.

### Solution

To solve the problem raised, an immunomodulatory composition comprising a set of autoantigenic peptides derived from synovial proteins and a method for the treatment or prevention of joint damage that corresponds to an antigen-specific therapy for subjects with Rheumatoid Arthritis (RA) and others rheumatological diseases consisting of tolerogenic dendritic cells (tDCs) pulsed with the autoantigenic peptides derived from synovial proteins to restore self-tolerance in in patients suffering RA and other rheumatological diseases by inhibiting the response of autoreactive T lymphocytes, are presented. The invention provides methods of use autoantigenic peptides. In certain embodiments, peptides in appropriate pharmaceutical carriers and formulated for administration, and methods of treatment of rheumatologic diseases.

This technology allows make the treatment compatible with the alleles of the main human histocompatibility complex (HLA) of a wide variety of patients in objective populations. The present invention can also be used in patients with Juvenile Idiopathic Arthritis (JIA) and Juvenile Rheumatoid Arthritis (JRA) and other related diseases.

### SUMMARY OF THE INVENTION

The present invention provides an immunomodulatory composition useful for the treatment or prevention of joint damage comprising at least a set of peptides possessing an amino acid sequence having at least 80%, 85% and 90% sequence identity with the peptides corresponding to SEQ ID NO:1, SEQ ID NO:2, SEQ ID NO:3 and SEQ ID NO:4 and a method for the treatment or prevention of joint damage comprising the steps of a) extract monocytes from a patient with a rheumatological disease; b) culture the monocytes extracted in the previous step in AIM-V medium with GM-CSF and IL-4; c) wash the monocytes and add dexamethasone; d) load the tDCs with the immunomodulatory composition comprising autoantigenic peptides; e) add MPLA; and f) incorporate the tDCs loaded with autoantigenic peptides into the patient. The present invention includes methods for the treatment or prevention of rheumatological disease comprising a wide range of tDCs performed by different protocols.

### BRIEF DESCRIPTION OF THE DRAWINGS

FIGS. 1A-1C. Reactivity of CD4+ T cells from patients with rheumatoid arthritis (RA) versus in vitro stimulation with autoantigenic peptides and control peptides. Peripheral blood mononuclear cells (PBMCs) from patients with RA (n=13) were stimulated with peptides derived from the following parental proteins: 3 peptides derived from Vimentin, such as, Vimentin-1, Vimentin-1 citrullinated (Cit), and Vimentin-2-Cit; 1 peptide derived from Calreticulin; 1 peptide derived from Fibronectin; and 1 peptide derived from Fibrinogen. Peptides derived from Hemagglutinin (HA) and Aggrecan were used as control peptides, also including the citrullinated form of the last. PBMCs in the absence of peptides stimulation and in the presence of beads coupled to anti-CD3 and anti-CD28 were used as negative and positive control, respectively. CD4+ T cell responses were evaluated by flow cytometry through the expression of the activation molecules: CD40L (FIG. 1A), CD69 (FIG. 1B) and CD25 (FIG. 1C), measured in the CD3+CD4+ T cell population. The response to each peptide is represented as a bar, calculated as fold increase of the percentage of CD4+ T cells expressing each activation molecule over the negative control, and its respective medians.

FIGS. 2A-2C. Reactivity of CD4+ T cells from patients with rheumatoid arthritis (RA) versus in vitro stimulation with autoantigenic peptides and control peptides. Peripheral blood mononuclear cells (PBMCs) from patients with RA (n=13) were stimulated with peptides derived from the following parental proteins: 3 peptides derived from Vimentin, such as, Vimentin-1, Vimentin-1 citrullinated (Cit), and Vimentin-2-Cit; 1 peptide derived from Calreticulin; 1 peptide derived from Fibronectin; and 1 peptide derived from Fibrinogen. Peptides derived from Hemagglutinin (HA) and Aggrecan was used as control peptides, also including the citrullinated form of the last. PBMCs in the absence of peptides stimulation and in the presence of beads coupled to anti-CD3 and anti-CD28 were used as negative and positive control, respectively. CD4+ T cell responses were evaluated by flow cytometry through the expression of the pro-inflammatory cytokines: IFN-γ (FIG. 2A), IL-17 (FIG. 2B) and TNF-α (FIG. 2C), measured in the CD3+CD4+ T cell population. The response to each peptide is represented as a bar, calculated as fold increase of the percentage of CD4+ T cells expressing each proinflammatory cytokine over the negative control, and its respective medians.

FIGS. 3A-3C Modulation of antigen-specific CD4+ T cell responses by tolerogenic dendritic cells (tDCs) treated with dexamethasone and monophosphoryl lipid A (MPLA-tDCs) and pulsed with autoantigenic peptides. MPLA-tDCs and MPLA-matured DCs (mDCs), as activation control, were generated from peripheral blood monocytes of RA patients, and pulsed with previously selected peptides or mixtures of them, using a peptide derived from Aggrecan, previously described in literature, as a control peptide. Subsequently, DCs were co-cultured with autologous CFSE-stained CD4+ T cells and, after 5 days, the percentage of proliferating CD4+ T cells (CFSElo) expressing IFN-γ (FIG. 3A), L-17 (FIG. 3B) or TNF-α (FIG. 3C) was evaluated by flow cytometry. Figure shows the median of a total of 5 experiments. To compare the expression of cytokines induced by each type of DCs, a T test was performed (*p<0.05).

FIGS. 4A-4C. Modulation of antigen-specific CD4+ T cell responses by tDCs generated using different protocols and pulsed with autoantigenic peptides. Two different protocols were used for tDCs generation from peripheral blood monocytes of RA patients: 1) DCs treated with Bay 11-082 (Bay-DCs); and 2) DCs treated with vitamin D3, dexamethasone and LPS (VD3-DCs); mDCs were used as control. DCs were pulsed with previously selected peptides or mixtures of them, using a peptide derived from Aggrecan, as a positive control. Then, DCs were co-cultured with autologous CFSE-stained CD4+ T cells and, after 5 days, 8 the percentage of proliferating CD4+ T cells (CFSElo) expressing IFN-γ (FIG. 4A), IL-17 (FIG. 4B) or TNF-α (FIG. 4C), was evaluated by flow cytometry. The percentage of decrease in the expression of proinflammatory cytokines by CD4+ T cells co-cultured with tDCs in comparison to mDCs was calculated. The figure shows the median of a total of 5 experiments.

### DETAILED DESCRIPTION OF THE INVENTION

Rheumatoid arthritis (RA) is an incurable, disabling autoimmune disease that decreases life quality and expectancy of around 73 million people worldwide. Current treatments achieve only temporary remission in a portion of RA patients and constraint to lifelong medication, with progressive loss of therapeutic efficiency and increase of costs. There is an urgent need for therapies that specifically restore tolerance towards self-antigens and thereby achieve permanent remission. A promising therapeutic approach is the administration of autologous tolerogenic dendritic cells (tDCs) generated from same patients and loaded with autoantigenic peptides.

The solution proposed in the present invention consists of an immunomodulatory composition comprising a new set of autoantigenic peptides derived from synovial proteins, and a method for the treatment or prevention of rheumatological diseases and joint damage, that corresponds to an antigen-specific therapy for subjects with rheumatoid arthritis and others rheumatologic diseases, consisting of tolerogenic dendritic cells (tDCs) pulsed with the autoantigenic peptides derived from synovial proteins to restore self-tolerance in patients with Rheumatoid Arthritis (RA), Juvenile Idiopathic Arthritis (JIA), Juvenile Rheumatoid Arthritis (JRA) and other related diseases by inhibiting the response of autoreactive T lymphocytes.

The present invention has identified by mass spectrometry new autoantigenic peptides derived from RA synovial proteins that are capable of binding to molecules encoded by Human Leukocyte Antigen (HLA)-DR alleles, specifically associated with RA (HLA-DRB1*0401, *0101, *0404, or *0405), in order to be presented by therapeutic tDCs. Selected peptides have also high promiscuity to other alleles, so they are not limited to the mentioned haplotypes.

To identify new autoantigenic peptides specific for rheumatoid arthritis, peptides bound to major histocompatibility complex (MHC)-class II molecules, either from DCs pulsed with synovial fluid from RA patients, DCs resident from synovial tissue from RA patients, or a cell-free system, were isolated and sequenced by mass spectrometry. After that, candidate peptides were analyzed using a bioinformatic platform and selected based on their theoretical affinity and promiscuity against HLA alleles. Selected peptides were synthesized and their reactivity in PBMC of patients with RA was evaluated, in order to analyze the percentage of autoreactive T cells against these autoantigens. In this manner, the aminoacidic sequence of the autoantigenic peptides of the invention was obtained.

In this approach it is essential that autoantigenic peptides be associated with HLA-DR molecules expressed in antigen presenting cells, such as dendritic cells, particularly to HLA-DR molecules with aminoacidic sequences characteristic of the target population, more specifically of patients with RA, so they can be susceptible to receiving the cellular therapy based on tDCs with autoantigenic peptides.

In this matter, the present invention consists of a composition comprising an autoantigenic peptides and an abbreviated protocol for the in vitro generation of human tDCs from peripheral blood monocytes, using dexamethasone as an immunomodulator and MPLA as an activator of tDCs.

The therapeutic approach developed in the present invention uses peptides derived from relevant autoantigens and associated to HLA-DR alleles prevalent in RA patients but not limited of these alleles.

A central element in the solution proposed here is related to the mechanism of action of the tDCs and the costs of therapy. Thus, infusion with tDCs pulsed with autoantigenic peptides restores the lost self-tolerance in RA patients that had been generated against those autoantigens, thus promoting the remission of the disease. For this, the therapy method can be performed as many times as necessary to achieve the goal.

In one aspect of the present invention, an immunomodulatory composition comprising at least a set of autoantigenic peptides derived from synovial proteins is proposed. Autoantigenic peptides comprise essentially amino acid derived sequences corresponding to synovial protein peptides. In some embodiments, the peptides essentially comprise an amino acid sequence corresponding to T cell epitopes.

The composition described in the present invention comprises at least a set of autoantigenic peptides derived from synovial proteins, such as vimentin, calreticulin, fibronectin and fibrinogen, in any of its possible combinations. From vimentin three peptides with different sequences were identified, named vimentin 1 and its citrullinated form, named vimentin 1-Cit, and citrullinated vimentin 2, named vimentin 2-Cit. In one embodiment of the present invention, the synovial proteins from which the peptides are derived are selected from the group consisting of vimentin 1, vimentin 2, fibronectin, fibrinogen, and calreticulin. In one embodiment of the present invention, the synovial proteins from which the peptides are derived are selected from the group consisting of vimentin 1, vimentin 2, fibronectin, fibrinogen, calreticulin, their processed, unprocessed, partially processed and mature forms. In another embodiment of the present invention, the autoantigenic peptides may be derived from the citrullinated forms of the synovial proteins selected from the group consisting of vimentin 1, vimentin 2, fibronectin, fibrinogen and calreticulin. In one embodiment of the present invention, the autoantigenic peptides are derived from proteins selected from the groups formed by vimentin 1 and vimentin 2 citrullinated forms. In one embodiment of the present invention, the autoantigenic peptides are derived from proteins selected from the groups formed by vimentin 1, vimentin 1-Cit, vimentin 2-Cit, fibronectin, fibrinogen and calreticulin. In a preferred embodiment, the synovial proteins from which the peptides are derived are selected from the group consisting of vimentin 1, vimentin 1-Cit vimentin 2-Cit, calreticulin, and fibrinogen.

In one embodiment of the present invention, the immunomodulatory composition for treatment or prevention of joint damage comprising peptides possessing an amino acid sequence having at least 90% or 95% sequence identity with the peptides corresponding to SEQ ID NO:1, SEQ ID NO:2, SEQ ID NO:3 and SEQ ID NO:4 is proposed. In another embodiment of the present invention, the immunomodulatory composition further comprises at least one peptide with an amino acid sequence having at least 90% or 95% sequence identity with the peptides corresponding to SEQ ID NO:5 and/or SEQ ID NO:6.

The terms “polypeptide,” “peptide,” and “protein” are used interchangeably herein to refer to polymers of amino acids of any length. The terms also encompass an amino acid polymer that has been modified; for example, disulfide bond formation, glycosylation, lipidation, phosphorylation, or conjugation with a labeling component. A polypeptide has a certain percent “sequence identity” to another polynucleotide or polypeptide, meaning that, when aligned, that percentage of bases or amino acids are the same when comparing the two sequences. Sequence similarity can be determined in a number of different manners. To determine sequence identity, sequences can be aligned using the methods and computer programs, including BLAST, available over the world wide web at ncbi.nlm.nih.gov/BLAST/. Another alignment algorithm is FASTA, available in the Genetics Computing Group (GCG) package, from Madison, Wis., USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Of particular interest are alignment programs that permit gaps in the sequence. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970).

Of interest is the BestFit program using the local homology algorithm of Smith and Waterman (Advances in Applied Mathematics 2: 482-489 (1981) to determine sequence identity. The gap generation penalty will generally range from 1 to 5, usually 2 to 4 and in many embodiments will be 3. The gap extension penalty will generally range from about 0.01 to 0.20 and in many instances will be 0.10. The program has default parameters determined by the sequences inputted to be compared. Preferably, the sequence identity is determined using the default parameters determined by the program. This program is available also from Genetics Computing Group (GCG) package, from Madison, Wis., USA.

Another program of interest is the FastDB algorithm. FastDB is described in Current Methods in Sequence Comparison and Analysis, Macromolecule Sequencing and Synthesis, Selected Methods and Applications, pp. 127-149, 1988, Alan R. Liss, Inc. Percent sequence identity is calculated by FastDB based upon the following parameters:

Mismatch Penalty: 1.00;

Gap Penalty: 1.00;

Gap Size Penalty: 0.33; and

Joining Penalty: 30.0.

In another embodiment, the immunomodulatory composition further comprises a pharmaceutically acceptable excipient including diluents, coadjuvants, buffering agents, surfactants, cosolvents, preservatives, sterile saline solution, phosphate buffered saline (PBS) solution, and Ringer-lactate solution, optionally supplemented with serum, preferably with autologous serum.

In an additional embodiment, the immunomodulatory composition further comprises an additional active pharmaceutical ingredient, including methotrexate, azathioprine, bucillamine, chloroquine, cyclosporin, doxycycline, hydroxychloroquine, intramuscular gold, leflunomide, levofloxacin and sulfasalazine; folinic acid, D-pencillamine, gold auranofin, gold aurothioglucose, gold thiomalate, cyclophosphamide and chlorambucil; tumor necrosis factor (TNF)-alpha inhibitors including infliximab, adalimumab, etanercept and golimumab; interleukin-1 inhibitors including anakinra; T-cell modulators including abatacept; B-cell modulators including rituximab; and interleukin-6 inhibitors including tocilizumab.

For therapeutic purposes, there is a convenience in the use of multiple antigens in the design of therapies based on tDCs, in order to favor a greater immuno-modulation, which is not restricted to a haplotype of the main histocompatibility complex (MHC) in particular.

The autoantigenic peptides described in this application are capable of inducing tolerance specifically directed to RA autoantigens when pulsed on tDCs, and furthermore they are compatible with the human MHC (HLA) alleles of a wide variety of patients.

The autoantigenic peptides described in this application are appropriate to load tDCs generated through different protocol and loaded with the peptides individually or as a mixture of them.

In one embodiment of the present invention, DCs were generated from monocytes isolated from peripheral blood by negative selection. Monocytes were cultured in serum-free AIM-V medium, supplemented with 100 to 1000 U/ml of GM-CSF and 100 to 1000 U/ml of IL-4 for 5 days at 35 to 40° C. and 5% CO2. At day 3, culture medium was replenished, and cells were incubated with dexamethasone at a final concentration of 1 to 10 μM. At day 4, cells were stimulated with 1 to 50 μg/ml of Monophosphoril Lipid-A (MPLA-tDCs). Unstimulated cells (DCs) and MPLA-matured DCs (M-DCs) generated in the absence of dexamethasone they can be used as controls of immature and mature DCs, respectively.

Autoantigenic peptides from synovial proteins will be selected from amino acid sequences having at least 90% or 95% sequence identity with the peptides corresponding to SEQ ID NO:1 to SEQ ID NO:6.

In a preferred embodiment, the composition of the present invention comprises a mixture of at least 4 peptides possessing an amino acid sequence having at least 90% or 95% sequence identity with the peptides corresponding to SEQ ID NO:1, SEQ ID NO:2, SEQ ID NO:3 and SEQ ID NO:4. Optionally, the composition can incorporate at least one peptide with an amino acid sequence having at least 90% or 95% sequence identity with the peptides corresponding to SEQ ID NO:5 and/or SEQ ID NO:6.

In related embodiments of the invention, the composition is administered to the subject in soluble or particulate form by injection, topical or nasal or oral application or any other relevant form of administration, for a period of time and in amounts that are suitably effective for suppress or reduce the autoimmune response of the T lymphocytes to the synovial protein peptides described in the present invention or to improve the symptoms associated with RA, other rheumatological diseases and reestablish self-tolerance in this patients.

In a preferred embodiment of the present invention, the composition comprising a set of autoantigenic peptides derived from synovial proteins are administered to the subject by therapy including loading said autoantigenic peptides possessing an amino acid sequence having at least 90% or 95% sequence identity with the peptides corresponding to SEQ ID NO:1, SEQ ID NO:2, SEQ ID NO:3 and SEQ ID NO:4 on tolerogenic dendritic cells (tDCs) of the patient. Optionally, autoantigenic peptides derived from synovial proteins are administered to the subject by therapy including loading said composition can incorporate at least one peptide with an amino acid sequence having at least 90% or 95% sequence identity with the peptides corresponding to SEQ ID NO:5 and/or SEQ ID NO:6 on tolerogenic dendritic cells (tDCs) of the patient.

In another embodiment, the composition comprising a set of autoantigenic peptides will be used to pulse tolerogenic dendritic cells, independent of the protocol used for their generation. These dendritic cells loaded with the autoantigenic peptides will be able to inhibit inflammatory responses of autoreactive CD4+T lymphocytes and modulate inflammatory immune response.

In another aspect, the present invention provides a method for the treatment or prevention of joint damage in a subject. This method is essentially based on generating an antigen-specific tolerogenic response to peptides derived from synovial proteins in the subject, treating or preventing joint damage.

A destructive autoimmune response has been implicated in various diseases such as rheumatoid arthritis (RA), in which the integrity of articular cartilage is destroyed by a chronic inflammatory process resulting from the presence of large numbers of activated lymphocytes and MHC class II expressing cells.

The antigen-specific tolerogenic response is achieved using a therapy method.

The method corresponding to antigen-specific therapy for subjects with rheumatoid arthritis and others rheumatological diseases consisting of the generation of tolerogenic dendritic cells (tDCs) pulsed with the autoantigenic peptides derived from synovial proteins to restore self-tolerance in patients with rheumatoid arthritis (RA), Juvenile Idiopathic Arthritis (JIA), Juvenile Rheumatoid Arthritis (JRA) and other related diseases, comprises the following stages:
(i) extracting monocytes from a patient with RA, JIA, JRA or another related disease;
(ii) culturing the monocytes extracted in the previous step in AIM-V medium (without serum), in the presence of 100 to 1000 U/ml of GM-CSF and 100 to 1000 U/ml of IL-4;
(iii) washing the monocytes and add dexamethasone until its differentiation to tolerogenic dendritic cells (tDCs);
(iv) loading the tDCs with a mixture comprising peptides possessing an amino acid sequence having at least 90% or 95% identity with the peptides corresponding to SEQ ID NO:1 to SEQ ID NO:4;
(v) adding 1 to 50 μg/ml of MPLA;
(vi) incorporating the tDCs loaded with autoantigenic peptides derived from synovial proteins in the patient.

Optionally, the mixture of autoantigenic peptides may additionally contain at least one peptide with an amino acid sequence having at least 90% or 95% sequence identity with the peptides corresponding to SEQ ID NO:5 and/or SEQ ID NO:6.

In one embodiment, step (iii) is performed on the third day of culture.

In one embodiment, step (iv) is performed on the fourth day of culture.

In one embodiment, step (v) is performed on the fourth day of culture.

In one embodiment, step (vi) is performed on the fifth day of culture.

In one embodiment, the step (vi) of the method is administrate via subcutaneous. In another embodiment, the step (vi) of the method is administrate via intravenously. In another embodiment, the step (vi) of the method is administrate via intraarticular. In another embodiment, in step (vi) of the method tDCs have a low level of co stimulatory molecules.

In another aspect, the present invention provides a method for treatment or prevention of joint damage comprising the following stages:
(i) extracting monocytes from a patient with RA, JIA, JRA or another related disease;
(ii) culturing the monocytes extracted in the previous step and differentiate tolerogenic dendritic cells;
(iii) loading the tDCs with a mixture comprising peptides possessing an amino acid sequence having at least 90% or 95% identity with the peptides corresponding to SEQ ID NO:1 to SEQ ID NO:4;
(iv) incorporating the tDCs loaded with peptides derived from synovial proteins in the patient.

Optionally, the immunomodulatory composition to be added in step (iv) further comprises at least one peptide with an amino acid sequence having at least 90% or 95% sequence identity with the peptides corresponding to SEQ ID NO:5 and/or SEQ ID NO:6.

In one embodiment, step (ii) of the method is through modulating agent.

In one embodiment, add MPLA activating agent before step (iii) of the method.

In one embodiment, the step (vi) of the method is administrate via subcutaneous. In another embodiment, the step (vi) of the method is administrate via intravenously. In another embodiment, the step (vi) of the method is administrate via intraarticular. In another embodiment, in step (vi) of the method tDCs have a low level of co stimulatory molecules.

The invention is further illustrated by the following examples, which are not meant to be construed in any as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the present invention and/or the scope of the appended claims.

### EXAMPLES

### Example 1

In order to evaluate the reactivity of CD4+ T cells in response to the autoantigenic peptides, peripheral blood mononuclear cells (PBMC), were obtain from rheumatoid arthritis patients through a density gradient isolation process. PBMC were cultured at final concentration of 106 cells/ml in 96 plate well for 5 days at 37° C. and 5% CO2, in presence of 3 peptides derived from vimentin, Vimentin-1, Vimentin-1 citrullinated, Vimentin-2 citrullinated, and autoantigenic peptides derived from Fibrinogen, Calreticulin and Fibronectin. Each autoantigenic peptide was evaluated individually. As a positive control, PBMC were stimulated with anti-CD3 and anti-CD28 antibody conjugated beads, and as a negative control, PBMC were incubated only in the presence of culture medium. Cells were re-stimulated with PMA (50 ng/mL) and ionomycin (1 μg/mL), in the presence of brefeldin-A (1 μg/mL) during the last 5 hours of culture, to evaluate by flow cytometry the expression of the activation molecules: CD25, CD69, CD40L, and the pro-inflammatory cytokines: IFN-γ, IL-17 and TNF-α.

### Results:

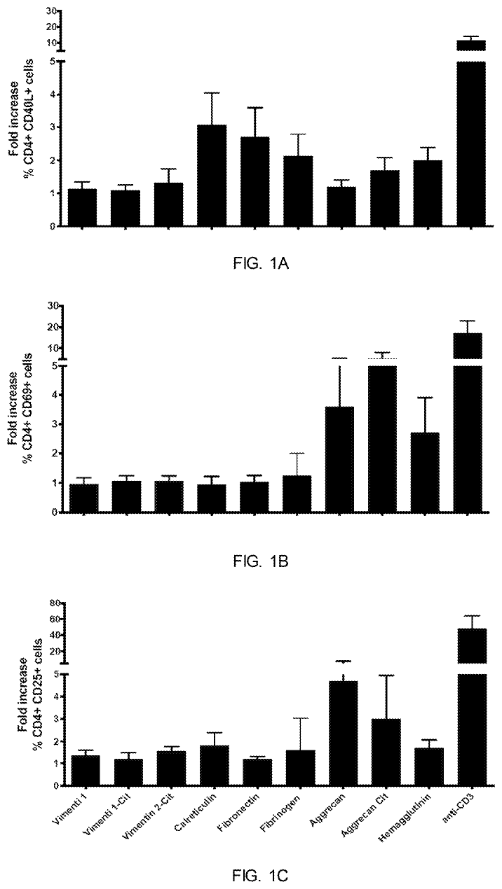

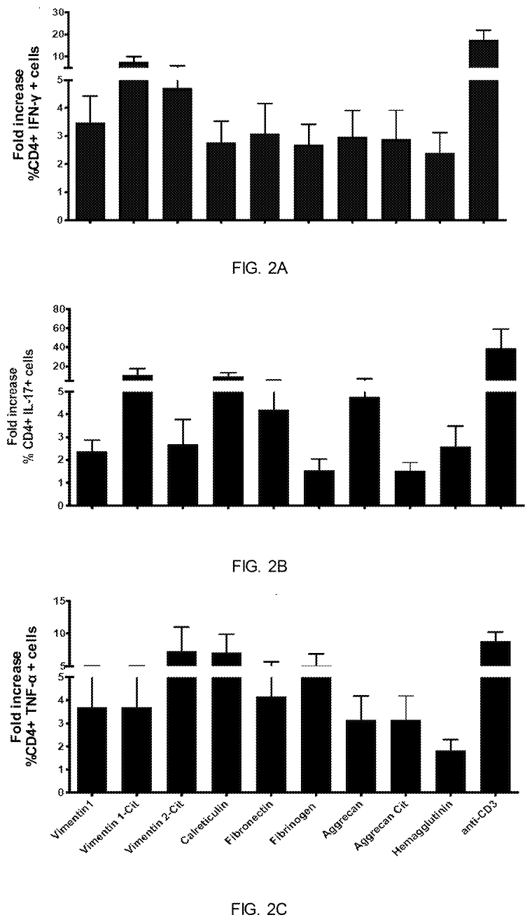

The results are expressed in times of increase in the percentage of CD4+ T cells expressing the activation molecules CD40L, CD69 and CD25 (FIGS. 1A, 1B and 10, respectively), in relation to T cells expressing said molecules in the absence of peptide. As can be seen in FIGS. 1A, 1B and 1C, there is a modest increase in the expression of activation molecules in CD4+T lymphocytes against autoantigenic peptides. However, as shown in FIGS. 2A, 2B and 2C, autoantigenic peptides induce a strong increase in the production of proinflammatory cytokines IFN-γ, IL-17 and TNF-α in CD4+ T cells over CD4+ T cells in the absence of stimulation.

These results indicate that the autoantigenic peptides that are processed and presented by dendritic cells present in the PBMC of patients with rheumatoid arthritis, are able to specifically stimulate CD4+T lymphocytes, allowing to identify those clones of autoreactive CD4+ T cells specific to said autoantigenic peptides, in patients with rheumatoid arthritis.

The presence of said autoreactive CD4+ T cell clones in response to the evaluated peptides present in peripheral blood of patients with rheumatoid arthritis, indicates that they are specific autoantigens of RA.

### Example 2

In order to evaluate the ability of tDCs to modulate antigen-specific CD4+ T cell responses, co-culture assays of tDCs pulsed with autoantigenic peptides and autologous CD4+ T cells from patients with RA were performed. DCs were generated from monocytes isolated from peripheral blood of patients with RA, cultured at a concentration of 2-3×10⁶ cells/ml in serum-free AIM-V medium in the presence of 500 U/mL of recombinant human interleukin-4 (rhIL-4) and recombinant human macrophage and monocyte colony stimulating factor (rhGM-CSF), for 5 days, at 37° C. and 5% CO2. For the induction of an activated tolerogenic profile, DCs were treated at day 3 of culture, with dexamethasone (1 μM), as immunomodulatory agent, and MPLA (1 μg/mL), as activation agent, during the following 24 hours, generating MPLA-tDCs. As control, mature DCs (mDCs) treated only with MPLA were used.

For carrying out the antigen-specific functional assays, on day 4 of generation of the DCs, 4 hours prior to their stimulation with MPLA, they were pulsed independently with the following autoantigenic peptides: 3 peptides derived from vimentin, Vimentin-1, Vimentin-1 citrullinated, Vimentin-2 citrullinated, or autoantigenic peptides derived from Fibrinogen, Calreticulin and Fibronectin. Likewise, they were also pulsed with selected mixtures of autoantigenic peptides at final concentration 100 μg/ml, or with a peptide derived from Aggrecan (100 μg/ml), as a positive control of the activation of the autologous CD4+ T cells. Un-loaded DCs were used as a basal activation control. The following mixtures were considered: Mix 1: Vimentin-1 and Vimentin-1 citrullinated; Mix 2: Vimentin-2 citrullinated and Calreticulin; Mix 3: Mix 1+Mix 2. At day 5 of culture, DCs were recovered, washed and co-cultured with CD4+ T cells, previously stained with the fluorescent molecule 5(6)-Carboxyfluorescein diacetate N-succinimidyl ester (CFSE), at a ratio of 1:2 (DC/T cells) in RPMI medium supplemented with 10% fetal bovine serum, for 5 days, at 37° C. and 5% CO2.

The effect of autoantigenic peptides to modulate effector CD4+ T cell responses of patients with RA, when are presented by tDCs generated using the protocol described in this application. As a positive control of activation of the autologous CD4+T lymphocytes, a peptide derived from Aggrecan was used, and un-pulsed DCs were used as control of basal activation. DCs were then co-cultured with CFSE-stained CD4+ T cells and, after 5 days of culture, the percentage of proliferating CD4+T lymphocytes, expressing IFN-γ, IL-17 and TNF-α, was evaluated by flow cytometry. In each case, the percentage of decrease in the expression of proinflammatory cytokines by CD4+ T cells co-cultured with tDCs in comparison to mDCs was calculated.

### Results:

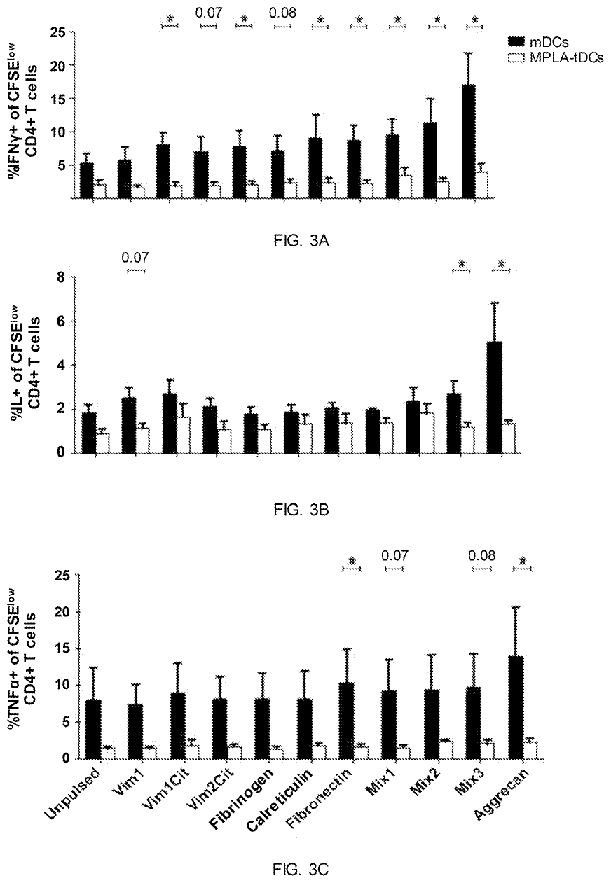

MPLA-tDCs pulsed with individual peptides or mixtures thereof tend to decrease the expression of the evaluated cytokines by CD4+T lymphocytes. In particular, in the case of IFN-γ, MPLA-tDCs pulsed independently with the peptides derived from Vimentin-1 citrullinated, Calreticulin and Fibronectin, in addition to the 3 mixtures tested, significantly reduced their expression in comparison to mDCs (FIG. 3A). In turn, it is observed that MPLA-tDCs pulsed with Vimentin 1, and MPLA-tDCs pulsed with the Mix of 3 peptides (Mix 3), significantly decreased the expression of IL-17 (FIG. 3B). Finally, MPLA-tDCs pulsed with peptide derived from fibronectin, significantly decrease the expression of TNF-α in CD4+T lymphocytes. A similar effect was observed in response to MPLA-tDCs pulsed with the peptides Mix named Mix-1 and Mix-3, which reduce the expression of TNF-α in comparison to mDCs (FIG. 3C).

Thus, the autoantigenic peptides described are capable of activating in an antigen-specific way effector CD4+ T cells from patients with RA, but more importantly, when the presentation is carried out by MPLA-tDCs, the result is a decrease in the response of these autoreactive effector CD4+ T cells. This has a great impact, since MPAL-tDCs administered in vivo could modulate autoreactive CD4+ T cell responses, being able to restore tolerance in 5 RA, as well as in other autoimmune diseases, for which autoantigenic peptides exist.

### Example 3

In order to evaluate the potential of autoantigenic peptides to load tolerogenic DCs generated from different protocols to modulate antigen-specific CD4+ T cell responses, co-culture assays of tDCs pulsed with autoantigenic peptides and autologous CD4+ T cells from patients with RA were performed. DCs were generated from monocytes according to the protocol described of example 2. To differentiate other tolerogenic dendritic cells types, DCs generated from monocytes were modulated with Bay-11-7982 an irreversible inhibitor of NF-κB (Bay-DCs), or modulated with Vitamin D3, dexamethasone and LPS (VD3-DCs). As control, mature DCs (mDCs) treated only with MPLA were used.

After this, the cells were pulsed with the autoantigenic peptides in the same way as an example 2, independently with the following autoantigenic peptides; 3 peptides derived from vimentin, such as Vimentin-1, Vimentin-1 citrullinated, Vimentin-2 citrullinated, or autoantigenic peptides derived from Fibrinogen, Calreticulin and Fibronectin, Aggrecan, as a positive control was used of the activation of the autologous CD4+ T cells, and un-pulsed DCs were used as a basal activation control. Besides mixtures were considered: Mix 1: Vimentin-1 and Vimentin-1 citrullinated; Mix 2: Vimentin-2 citrullinated and Calreticulin; Mix 3: Mix 1+Mix 2.

tDCs were then co-cultured with CFSE-stained CD4+ T cells and, after 5 days of culture, the percentage of proliferating CD4+T lymphocytes, expressing IFN-γ, IL-17 and TNF-α, was evaluated by flow cytometry. In each case, the percentage of decrease in the expression of proinflammatory cytokines by CD4+ T cells co-cultured with tDCs in comparison to mDCs was calculated.

### Results

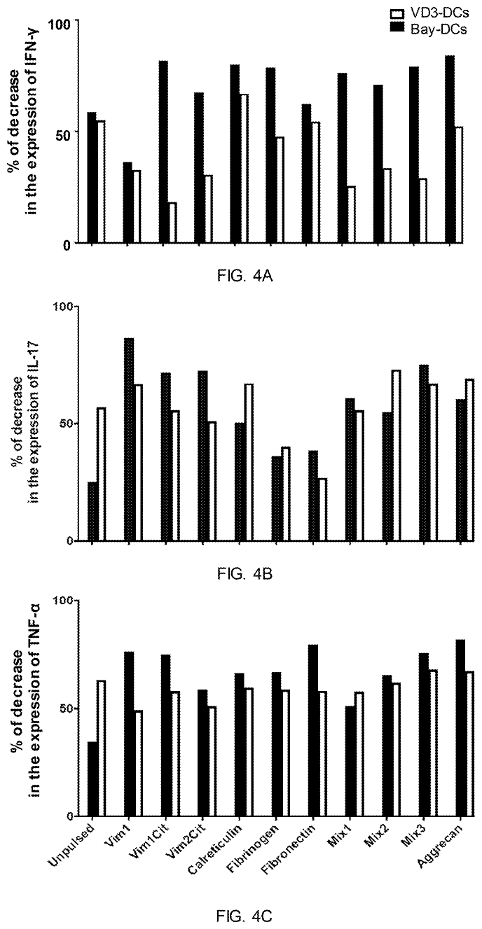

Bay-DCs pulsed independently with the autoantigenic peptides derived from Vimentin-1 citrullinated, Calreticulin and Fibronectin, in addition to the 3 mixtures tested, significantly reduced expression of IFN-γ in comparison to the mDCs with individual peptides or mixtures, Bay-DCs tend to decrease the percentage of expression of IFN-γ by CD4+T lymphocytes in a greater proportion than the VD3-DCs, however, both cell types decrease this cytokine in a specific way (FIG. 4A). When IL-17 was evaluated, VD3-DCs and Bay-DCs pulsed with Vimentin 1, Vimentin 1-Cit, Vimentin 2-Cit, Calreticulin and Fibrinogen, or pulsed with Mix 1, Mix or Mix, showed a high percentage of decrease 1L-17 expression by CD4+T lymphocytes (FIG. 4B).

Likewise, a decrease the expression of TNF-α evaluated by CD4+T lymphocytes was observed using both tDCs protocols (FIG. 4C), in response to independent autoantigen peptides or mixes of them.

Independent of the protocol used to generate the tDCs, they demonstrate to decrease the expression of pro-inflammatory cytokines with respect to the expression induced when the peptides are presented by mDCs to the T lymphocytes.

1. An immunomodulatory composition comprising one or more peptides having an amino acid sequence of at least 90% or 95% sequence identity to an amino acid sequence as set forth in SEQ ID NO:1, SEQ ID NO:2, SEQ ID NO:3, SEQ ID NO:4, SEQ ID NO:5, or SEQ ID NO:6.

2-4. (canceled)

5. An immunomodulatory composition comprising antigen-presenting cells, wherein the antigen-presenting cells are loaded with one or more peptides as defined in claim 1.

6. The immunomodulatory composition according to claim 5, wherein the antigen-presenting cells are tolerogenic dendritic cells.

7. The immunomodulatory composition according to claim 5, further comprising a pharmaceutically acceptable excipient selected from a group consisting of diluents, coadjuvants, buffering agents, surfactants, cosolvents and preservatives.

8. The immunomodulatory composition according to claim 7, wherein the pharmaceutically acceptable excipient is selected from a group consisting of sterile saline solution, phosphate buffered saline (PBS) solution, and Ringer-lactate solution.

9. The immunomodulatory composition according to claim 5, further comprising an additional active pharmaceutical ingredient.

10. The immunomodulatory composition according to claim 9, wherein the additional active pharmaceutical ingredient selected from the group consisting of disease modifying anti-rheumatic drugs (DMARDs) including methotrexate, azathioprine, bucillamine, chloroquine, cyclosporin, doxycycline, hydroxychloroquine, intramuscular gold, leflunomide, levofloxacin and sulfasalazine; folinic acid, D-pencillamine, gold auranofin, gold aurothioglucose, gold thiomalate, cyclophosphamide and chlorambucil; tumor necrosis factor (TNF)-alpha inhibitors including infliximab, adalimumab, etanercept and golimumab; interleukin-1 inhibitors including anakinra; T-cell modulators including abatacept; B-cell modulators including rituximab; and interleukin-6 inhibitors including tocilizumab.

11. (canceled)

12. (canceled)

13. A method for treatment or prevention of a rheumatological disease in a subject in need thereof, comprising administering to the subject a therapeutically effective amount of an immunomodulatory composition as defined in claim 1.

14. The method according to claim 13, wherein the rheumatological disease is Rheumatoid Arthritis (RA), Juvenile Idiopathic Arthritis (JIA) or Juvenile Rheumatoid Arthritis (JRA).

15. A method for treatment or prevention of joint damage comprising following steps:
(i) extracting monocytes from a patient with Rheumatoid Arthritis (RA), Juvenile Idiopathic Arthritis (JIA), Juvenile Rheumatoid Arthritis (JRA) or another related disease;
(ii) culturing the monocytes extracted in step (i), washing and differentiating to tolerogenic dendritic cells (tDCs);
(iii) loading the tDCs with the immunomodulatory composition of claim 1 for obtaining loaded tDCs; and
(iv) administering the loaded tDCs of step (iii) to the patient.

16. The method according to claim 15,
wherein a serum-free media in the presence of 100 to 1000 U/ml of granulocyte-macrophage colony-stimulating factor (GM-CSF) and 100 to 1000 U/ml of interleukin-4 (IL-4) is used for culturing monocytes extracted.

17. (canceled)

18. The method according to claim 15, wherein the loaded tDCs are administered to the patient via subcutaneous injection, intravenous injection, or intraarticular injection.

19. (canceled)

20. (canceled)

21. The method according to claim 15, wherein the tDCs administered to the patient have a low level of co-stimulatory molecules.

22. The immunomodulatory composition according to claim 8, wherein said composition further comprises serum or autologous serum.

23. The method according to claim 15, wherein the step (iii) further comprises adding 1 to 50 μg/ml of monophosphoryl lipid A (MPLA) to the loaded tDCs.


SEQUENCE LISTING (6 sequences)

SEQ ID NO: 1 - protein, 15 aa, Homo sapiens
SFRQDVDNASLARLD

SEQ ID NO: 2 - protein, 15 aa, Homo sapiens
  Cit (3)..(13)
SFXQDVDNASLAXLD

SEQ ID NO: 3 - protein, 15 aa, Homo sapiens
  Cit (5)..(5)
ELNDXFANYIDKVRF

SEQ ID NO: 4 - protein, 14 aa, Homo sapiens
GGGYVKLFPNSLDQ

SEQ ID NO: 5 - protein, 13 aa, Homo sapiens
GTYSKASTPNGYD

SEQ ID NO: 6 - protein, 17 aa, Homo sapiens
GFMKEINLAFDSSSVVV